(19) United States
(12) Patent Application Publication
(10) Pub. No.: US 2026/0240416 A1
(43) Pub. Date: Aug. 20, 2026

(54) ENDOSCOPE SYSTEM HAVING A SINGLE-USE PART AND A REUSABLE PART

(71) Applicant: Konstantin Bob, Weinheim (DE)

(72) Inventor: Andreas Gründl, Starnberg (DE)

(73) Assignee: Konstantin Bob, Weinheim (DE)

(21) Appl. No.: 19/164,824

(22) PCT Filed: Mar. 11, 2024
(86) PCT No.: PCT/EP2024/056391

§ 371 Date: Sep. 12, 2025

(30) Foreign Application Priority Data
Mar. 15, 2023 (DE) 10 2023 106 457.6

(51) Int. Cl. A61B 1/00; A61B 1/005; A61B 1/015

(52) U.S. Cl. CPC A61B 1/00105; A61B 1/00103; A61B 1/00128; A61B 1/0057; A61B 1/015

(57) ABSTRACT

A single-use part and a reusable part for an endoscope system that includes: a proximal operating portion, a flexible shaft; a distal endoscope portion, which is connected to a distal end portion of the flexible shaft; and a distal endoscope head. The operating portion is divided into a single-use operating portion and a reusable operating portion. The single-use part includes a single-use drivetrain portion, which connects the single-use operating portion to the distal endoscope portion, and a proximal single-use coupling portion, which is mounted so as to be displaceable in the direction of the shaft axis and forms, at its proximal end, a single-use engagement element which is provided for coupling to a reusable drivetrain portion. The reusable part includes an electric drive, for controlling the endoscope, and the reusable drivetrain portion, which is proximally connected to the electric drive and distally has a longitudinally displaceable reusable engagement element.

The present disclosure relates to a single-use part and a reusable part of an endoscope system comprising a proximal operating portion, a flexible shaft, a distal endoscope portion connected to a distal end portion of the flexible shaft, and a distal endoscope head. The operating portion is divided into a single-use operating portion with a single-use drivetrain portion and a reusable operating portion with a reusable drivetrain portion, which are optionally coupleable with each other.

### BACKGROUND OF THE DISCLOSURE

Endoscopes are medical working devices for the visual exploration of cavities in a patient's body. They basically have optical devices on the distal, i.e. facing a user or facing a patient's body, endoscope head and a flexible or bend-proof (rigid) shaft which connects the endoscope head to a proximal operating portion or endoscope handle. The proximal, i.e. facing a user, operating portion or endoscope handle is an assembly configured for use with an endoscope and connected or connectable thereto in order to control the endoscope. Typically, the endoscope furthermore has a working channel that extends from the proximal operating portion through the shaft to a working channel exit formed on the endoscope head and allows for the extracorporeal insertion and use of a medical instrument such as forceps, scissors, needle, snare, knife and the like. Many endoscopes also have deflections, i.e. a steerable shaft portion located between the shaft and the endoscope head to allow steering of the endoscope in a patient cavity. Furthermore, endoscopes may optionally be provided with additional capabilities that require additional control options, such as an endoscope known from DE 10 2018 110 620 A1, which discloses a folding mechanism or a tilt head for narrowly folding down an endoscope head.

Such endoscopes can be controlled either via a complex, manual control mechanism, which has an operating part/handle with handwheels, buttons and the like, from which a control force is transmitted, for example via pinions or drums, to control wires of the endoscope, which run through the endoscope shaft and transmit the control force to elements to be controlled in the endoscope shaft. Alternatively, such endoscopes can be controlled via a control mechanism that has an operating part/handle with electric motors that are activated and controlled in order to apply the control force to the control wires of the endoscope. An example of such an endoscope can be found in WO 92/01 414 A1.

Endoscope systems are therefore very complex, expensive products. Accordingly, endoscopes and endoscope handles are often configured as reusable products. On the other hand, endoscopes are inserted into patient cavities during operations and examinations. The risk of contamination of the endoscope and the patient concerned is therefore high. Cleaning, disinfection and/or sterilization of endoscopes is time-consuming and expensive and prone to errors due to the complex geometry of endoscopes. In view of this aspect, it is advantageous to configure endoscopes as single-use products.

U.S. Pat. No. 8,449,456 B2 discloses an endoscope with a shaft portion intended for insertion into a patient and which has a deflection, i.e. a bendable portion, a control wire attached to it and a spindle drive attached to the wire. The shaft portion is detachably connected to a drive unit, which has a motor, via a manually operable connection with a pin engaging in a rotating ring. The motor is coupleable to the spindle drive of the shaft via a spindle shaft and a coupling in order to transmit driving force of the motor to the spindle, to convert it into a tractive force, and to transmit it to the deflection via the wire in order to bend it, and to pivot an endoscope head in at least one pivot axis. However, the connecting mechanism between the shaft portion and the drive unit is complex and therefore expensive and prone to contamination. Furthermore, establishing the corresponding connection is time-consuming for the user.

### SUMMARY OF THE DISCLOSURE

The object underlying the disclosure is to avoid or reduce disadvantages of the prior art. In particular, an endoscope system with a single-use part and a reusable part as well as a corresponding single-use part and reusable part is to be provided, which are easily and safely coupleable with and separable from each other.

The object is solved by a single-use part of an endoscope system according to claim 1, a reusable part of an endoscope system according to claim 9, and an endoscope system according to claim 15. Advantageous embodiments are the subject matter of the dependent claims.

More specifically, the object is solved by a single-use part of an endoscope system comprising an endoscope and a single-use operating portion of a proximal operating part (e.g. handle) which is configured to be selectively coupled to a reusable part of the proximal operating part or handle described in more detail later. The endoscope has a flexible shaft configured for insertion into a body opening of the patient, is connected to the single-use operating portion of the operating part or handle, and extends therefrom along a shaft axis in the distal direction; a distal endoscope portion connected to a distal end portion of the flexible shaft; and a distal endoscope head forming a distal front side of the distal endoscope portion or being attached thereto. Furthermore, the single-use part has at least one single-use drivetrain portion connecting the single-use operating portion to the distal endoscope portion in order to control the distal endoscope portion. The at least one single-use drivetrain portion has at least one single-use coupling portion, which is a part of the operating part or handle, is slidably (preferably rotationally fixedly) mounted in the operating part or handle in the direction of the shaft axis and has a single-use engagement element configured at its proximal end, which is configured for axially fixed coupling (i.e. transmitting a pulling and/or pushing movement or being immovable relative to each other in the direction of the shaft axis) with a reusable drivetrain portion of the reusable operating portion. More precisely, the single-use engagement element forms a free end of the single-use drivetrain portion in an uncoupled state.

In other words, a single-use part of an endoscope system with a controllable endoscope and a single-use operating portion proximally connected thereto is provided, comprising a single-use coupling portion (connecting mechanism) which forms a proximal end of a single-use drivetrain portion (i.e. a power transmission cable) and which is slidable in distal and proximal directions. More specifically, the single-use coupling portion forms a part of the single-use drive train, which connects the engagement element (in particular the snap connector) to control cables of the endoscope for controlling the distal endoscope portion. The single-use operating part, preferably the entire single-use part, is in particular a passive assembly, i.e. without its own electrical drives configured. The single-use part has a single-use drivetrain portion, which is a (purely) mechanical power transmission cable that connects a proximal connecting element of the single-use operating part, i.e. the single-use engagement element of the single-use coupling portion, via control cables of the endoscope to controllable portions on the distal endoscope portion. In other words, the single-use drivetrain portion is configured to transmit a driving force to drivable mechanisms (e.g., a deflection or other mechanisms mentioned below) in a distal endoscope portion (i.e., in an endoscope tip) for controlling the distal endoscope portion.

Furthermore in other words, integrated in the single-use drive train, in particular in its proximal end portion, is the single-use coupling portion which has an engagement element, i.e. an element which has an optionally elastic undercut or an optionally elastic protrusion and which is configured for an axially fixed, engaging connection with a reusable engagement element. That is, the single-use coupling portion (at least its proximal end or the single-use engagement element) is configured for mechanical engagement, in particular snap-in connection/snap-in engagement, with the reusable engagement element. The single-use engagement element preferably forms a proximal end of the single-use drivetrain portion. The single-use coupling portion is mounted so that it can slide axially or in a longitudinal direction (i.e. in the direction of the shaft axis). This enables a particularly simple and cost-effective construction of the single-use part and particularly simple, largely automated coupling with the reusable part, which reduces the possibility of operating errors on the part of the user.

Preferably, the single-use engagement element for the snap-in engagement has a radially elastically deformable spring element. Preferably, the single-use engagement element of the single-use coupling portion is a rotationally symmetrical component (e.g. a cylinder) which carries an elastic snap-fit element, in particular an annular clutch spring, on an outer or inner circumferential wall. The clutch spring may be a BalSeal-spring, for example, whose windings run diagonally in such a way that they fold in under a radial load.

Throughout the disclosure, terms such as ‘axial’, ‘circumferential direction’ and ‘radial’ always refer to the shaft axis, unless explicitly stated otherwise. The shaft axis is a central axis of the flexible shaft and of the distal endoscope portion. The shaft may be rigid or flexible. The endoscope head may also have lighting equipment and/or a flushing device and/or other elements connected to associated lines. Distal refers to a direction facing the patient/in which the endoscope is inserted into a cavity of the patient. Proximal refers to a direction of the endoscope facing the user/the attending physician or surgical assistant. ‘Single-use’ in the present disclosure refers to components which are assigned to the single-use part, and ‘reusable’ in the present disclosure refers to components which are assigned to the reusable part, in each case in relation to a state in which the single-use part and the reusable part are provided separately from each other.

In particular, the proximal operating portion is a manually operable endoscope handle (i.e. a handle for use with an endoscope to control it). The single-use operating portion has, for example, components which may become contaminated when the endoscope is used and/or which are subject to high wear or are frequently damaged, such as, for example, a processor port, a (Luer) connection (i.e. an access to a working channel), and an air-water button.

The single-use drivetrain portion is in particular a mechanical and/or electrical (preferably purely mechanical) connection, which is configured to transmit a driving force (of an electrical drive of the reusable part), which is applied to the single-use coupling portion (more precisely, to its single-use engagement element), to the distal endoscope portion or to controllable mechanisms arranged there. In particular, the single-use drivetrain portion has one or more control cables, which are part of the endoscope and are configured to drive drivable or controllable portions on the distal endoscope portion. The single-use coupling portion of the single-use operating part may transmit a driving force, in particular in the form of a pushing or pulling movement, to the control cable.

In particular, a plurality of single-use drivetrain portions are provided, which are each provided substantially independently of each other, may be controlled substantially independently of each other, and which each have (exactly) one single-use coupling portion, (exactly) one control cable and preferably (exactly) one connecting component for connecting the control cable and the single-use coupling portion, e.g. a cross connector. The connecting component is in particular part of the single-use operating portion and accommodated therein. In this case, each of the single-use drivetrain portions is coupled or coupleable with (exactly) one reusable drivetrain portion.

The distal endoscope portion preferably has a deflection as a first controllable portion, which is controllable in such a way that it may be actively deflected in at least (preferably exactly) one lateral direction. This allows the endoscope to be navigated within the cavities of the patient. Furthermore, the distal endoscope portion preferably has a folding mechanism or a tilt head as a second controllable portion, which is arranged distally from the deflection and may be bent at least (preferably exactly) in one different or the same lateral direction as the deflection, namely with a narrower radius of curvature than the deflection. This allows optics aligned on the distal endoscope head in the shaft direction or a working channel of the endoscope opening there to be folded down for a shoulder view. A rotation mechanism is further preferably provided as a third controllable portion, in particular between the deflection and the flexible shaft, which enables the distal endoscope portion to be rotated about its shaft axis. This allows the deflection and/or the folding mechanism or tilt head to be rotated. This makes it particularly easy to set these up without restricting the viewing direction of the endoscope. Preferably, the deflection and/or the tilt head and/or the folding mechanism and/or the rotation mechanism may each be controlled by a separate control cable, in particular substantially independently of each other.

Preferably, the single-use operating portion forms a first (distal) housing portion in which the at least one single-use coupling portion is slidably mounted in a longitudinal direction (i.e. in the direction of the shaft axis) and a corresponding displacement movement in at least one direction along the shaft axis, preferably in both directions along the shaft axis, is limited by a stop in each case.

In other words, the single-use drive train, in particular the single-use coupling portion, has at least one, preferably two (one in the proximal and one in the distal direction) stop portion(s) which abut against housing-fixed portions of the single-use operating portion in order to limit a movement of the at least one single-use coupling portion in the longitudinal direction such that it can only move in a predetermined axial range. In particular, the at least one stop is arranged and configured such that it forms an abutment to establish or release coupling or decoupling of the single-use coupling portion (in particular when establishing or releasing a snap-in engagement with the single-use coupling portion).

Advantageously, it is thus possible to apply a pressure and/or a tension to the single-use coupling portion for coupling or decoupling the single-use coupling portion, which counteracts a coupling force (e.g. an elastic restoring force of a snap-fit element), as described in more detail later. In this way, a particularly simple, robust coupling and/or decoupling process can be provided. Preferably, for each single-use drivetrain portion, a first stop is provided in a first direction (in particular in the distal direction) for coupling the single-use coupling portion, and a second stop is provided in a second direction (in particular in the proximal direction) (opposite to the first direction) for uncoupling the single-use coupling portion.

One of the at least one stop for limiting the longitudinal movement of the single-use coupling portion may preferably be provided by the single-use engagement element (e.g. by the rotationally symmetrical component), which is arranged at the proximal end of the single-use coupling portion. For example, at least one single-use engagement element (i.e. the one single-use engagement element or one of the single-use engagement elements) of the single-use coupling portion may have a surface facing in the longitudinal direction (e.g. a front side of a cylindrical engagement element), which serves as a stop portion. This means that no separate component has to be provided for the stop portion and the number of parts required may be reduced, thereby reducing costs and assembly work. A further stop may be provided on a proximal portion of the single-use coupling portion, e.g. by a cross connector described later.

The expression ‘one of the at least one’ element, portion or the like describes in each case the one element, portion, etc., or one of the elements, portions, etc., if several of them are provided.

Preferably, one or more separate adjustment stop component(s) may be provided on the single-use coupling portion and/or on the single-use housing portion in such a way that it/they may be adjusted in its/their axial position. For example, one or more of the adjustment stop component(s) may be an adjustment nut which may be screwed onto or into the single-use coupling portion and/or the single-use housing portion in the longitudinal direction. This allows the position of the stop to be adjusted in an advantageous manner. Preferably, the longitudinal movement of each drive train (i.e. the at least one single-use drive train or several single-use drive trains) is limited in exactly one of the longitudinal directions (i.e. either in the distal or proximal direction) by an adjustment stop component(s) and in the other one of the longitudinal directions by the single-use engagement element.

Preferably, the at least one stop is provided such that the single-use engagement element is located in its most proximal position at least partially, further preferably completely, within the distal single-use housing portion. In other words, the at least one stop is arranged in such a way that movable parts of the single-use drivetrain portion, in particular the single-use coupling portion or even the single-use engagement element, are arranged largely and preferably completely within the single-use housing portion in each displacement position.

As a result, the components of the single-use part, in particular of the single-use operating part, are particularly well protected. In particular, the at least one single-use coupling portion may be mounted loosely, i.e. unpowered and possibly substantially unbraked, in the single-use housing portion without it protruding from the single-use housing portion and being damaged in the process. This means that the single-use coupling portion can be actuated particularly easily and with low friction.

Preferably, a partition wall or frame is provided, which is arranged within the single-use housing portion and extends transversely to the shaft axis. The partition wall or the frame may form a bearing direction for the longitudinal displacement of the single-use coupling portion. In particular, the single-use coupling portion may extend through the partition wall or frame. Furthermore, the partition wall or frame may form stop surfaces as a part of one or preferably both of the at least one stop for limiting the longitudinal movement of the single-use coupling portion. In other words, the partition wall may form an abutment for the at least one stop portion of the single-use coupling element and may be dimensioned such that it absorbs forces generated during coupling and/or decoupling of the single-use coupling portion (with the reusable coupling element).

This can provide a particularly simple, cost-effective and stable framework for the single-use housing portion. The framework may support a simple, inexpensive casing to form the single-use housing portion.

Preferably, the partition wall divides the single-use housing portion into a distal chamber in which the single-use coupling portion is connected to proximal ends of control cables, and a proximal chamber in which the single-use engagement element is located. In other words, the single-use housing portion protrudes distally beyond the partition wall to form the proximal chamber or a proximal sleeve. This allows the single-use coupling portion to be received and protected within the single-use housing portion, wherein the single-use engagement element is disposed in the proximal chamber. Furthermore, particularly sensitive components of the single-use part, in particular of the single-use operating part, such as connections (connecting components) for the control cables, a tension roller described later and the like, may be arranged in the distal chamber and can be additionally protected from dust etc.

Preferably, the single-use housing portion further forms a proximal front side in which at least one opening for insertion of the at least one reusable-drive train is configured.

Further preferably, the single-use housing portion forms a plug portion which, in particular with respect to the shaft axis, starts at a radial side of the proximal front side of the single-use housing portion and extends in the proximal direction from the single-use housing portion. The plug portion is preferably configured for connection to electrical (for power supply and control) and fluidic lines of the reusable part. In particular, the plug portion has a processor port which is configured to connect the electrical and fluidic lines of an external endoscope control unit. Furthermore, fluid lines are preferably guided in the plug portion or configured integrally therewith in order to connect the processor port with fluid lines which run through the flexible shaft to the endoscope head.

Preferably, the plug portion forms an electrical single-use connection on its proximal front surface, in particular an electrical plug or socket. The electrical single-use connection is configured in particular for connection to an electrical reusable connection, in particular an electrical socket or plug, of the electrical reusable connection. Preferably, the processor port is provided on a radial side (also referred to as a lateral outer edge) of the plug portion. In particular, the processor port is provided at a proximal end region of the radial side/lateral outer edge. As a result, electrical lines leading from the processor port to the electrical single-use connection may be particularly short.

Preferably, the plug portion is configured to be substantially plate-shaped and has a possibly rounded radial outer surface, two lateral outer edges (i.e. two radial sides), which point radially outward, and the proximal front surface. A single-use guide surface is preferably provided opposite the radial outer surface and in particular diagonally facing the proximal front side of the single-use housing portion (i.e. arranged substantially perpendicular thereto). The single-use guide surface is preferably flat/planar. In other words, the single-use guide surface and the proximal front side of the single-use housing portion may be substantially perpendicular to each other or may form two sides of a substantially cuboid recess configured to receive the reusable part. The single-use guide surface may be configured for contact with a corresponding reusable guide surface of the reusable part and may thus provide a relatively large contact surface of the two parts. This enables particularly stable, secure guidance and support of the reusable part and single-use part when they are coupled together or inserted into each other in the longitudinal direction like drawers. Optionally, a guide rail may be configured on one or both lateral outer edges.

Preferably, a protrusion is configured on a lateral outer edge, which is arranged on one side of the single-use guide surface and protrudes substantially perpendicularly thereto to provide an air-water button described later. That is, the protrusion may define a further side of the substantially cuboid recess. This may further improve the guidance of the reusable part during coupling. Optionally, an additional latch element (e.g. a latching structure, such as a trough, or a spring element) may be provided on a side of the protrusion facing the single-use guide surface, which provides the air-water button, wherein the latch element is configured to latch with a matching latch element on the reusable part. This can provide audible and/or tactile feedback when the protrusion is correctly positioned on the reusable part. Preferably, a switch may further be provided, e.g. integrated into one of the latching openings or the latching structure or provided by the electrical reusable switch, which recognizes complete coupling of the single-use housing portion and the reusable housing. The control/computer unit may preferably automatically trigger a calibration and/or coupling of the single-use and reusable drivetrain portions when the switch is closed.

Preferably, the single-use operating portion forms at least one latching nose configured for snap-in engagement and has a predetermined breaking point configured to break when the snap-in engagement is released. This may reduce the risk of a single-use operating portion being used more than once and may therefore reduce the risk of contamination during an operation. Preferably, the at least one latching nose is provided on the proximal front side of the single-use housing portion and protrudes in the proximal direction therefrom.

Further preferably, one or more guide holes or guide pins are provided, preferably on the proximal front side of the single-use housing portion and/or on the proximal front side of the plug portion. In particular, one or more matching guide holes or guide pins are provided on the reusable part at correspondingly opposite points and are configured to engage (for guidance in the longitudinal direction) with the guide hole(s) or pin(s) of the single-use operating part. This enables particularly precise guidance of the single-use part and reusable part when they are coupled, in particular when the electrical single-use connection is connected to the electrical reusable connection.

Preferably, each of the at least one single-use coupling portion (i.e. the single-use coupling portion or several coupling portions) is connected at its distal end to a control cable via a cross connector, wherein the cross connector compensates for a radial offset between the control cable and the respective single-use coupling portion. This enables greater flexibility in the arrangement and configuration of the single-use coupling portions and allows the routing of the control cable to be optimized. In other words, the cross connector is a connecting component that extends transversely to the shaft direction and is connected on one side to the associated single-use coupling portion and on the other side to the associated control cable.

Preferably, one of the at least one control cable is a shear-resistant and/or low-stretch tube in which a fluid channel is configured. Further preferably, the cross connector forms a connection line which is connected at its first end to the fluid channel and at its second end forms a fluid connection for a flexible roll hose of the single-use operating part. Shear-resistant in the sense of the present disclosure means that the corresponding control cable is suitable for transmitting compressive forces. Low-stretch in the sense of the present disclosure means that the corresponding control cable is suitable for transmitting tensile forces and that any stretching of the control cable is negligible.

Preferably, the control cable, which forms the fluid channel, is connected to the endoscope head and opens at its distal front side. In particular, a distal end of the fluid channel is connected to a cleaning nozzle for cleaning the camera or the illumination of the endoscope head. In this way, the number of cables and lines routed through the flexible shaft can be reduced and the diameter of the flexible shaft can be minimized. Preferably, the control cable that forms the fluid channel is a control cable for controlling a controllable endoscope portion arranged furthest distally, in particular the tilt head or the folding mechanism.

The roll hose may be a flexible tube which can be placed in a loop and can be moved/walked/rolled to allow longitudinal movement of the control cable connected to it. The fluid connection between the respective control cable and the roll hose may be provided by a line configured within the cross connector connecting the respective control cable to the respective single-use coupling portion. That is, the line of the cross connector may open into the fluid channel in the control cable at one end and be fluidly connected to the roll hose at its other end. This means that the number of parts can be further reduced. Alternatively, an additional connection element may be provided separately from the cross connector, which connects the roll hose to the fluid channel in the control cable.

If applicable, a single-use part of an endoscope system (and possibly the corresponding system with the single-use part and a reusable part described above) with a proximal operating portion, a flexible shaft, a distal shaft portion (e.g. deflection), which can be controlled via at least one control cable, and an endoscope head can be claimed independently, wherein one of the at least one control cable configures a fluid channel (in particular according to the above description), which opens in the endoscope head and which forms a fluid connection at a proximal end, e.g. via a cross connector described above, for a flexible roll hose. Such a single-use part or endoscope system may further have other features described in the present disclosure.

Preferably, a first single-use drivetrain portion is provided, which has a first control cable (e.g. the control cable for controlling the deflection or a deflection control cable), and a second single-use drivetrain portion is provided, which has a second control cable (e.g. a control cable for controlling the tilt head/the folding mechanism or a tilt head control cable, possibly with the fluid channel configured therein). Further preferably, the first control cable is a shear-resistant and/or low-stretch tube in which the second control cable is accommodated. In other words, the first control cable may be a shear-resistant and/or low-stretch hollow pipe/tube, inside which the second control cable is guided. This makes it possible to further reduce the number of strands guided through the flexible shaft and to further minimize its diameter.

Preferably, one of the at least one control cable (in particular a third control cable or rotation mechanism control cable) runs through the flexible shaft and is connected at its distal end to a tension cable, preferably via a distal transducer or a distal transmission element of the rotation mechanism. In particular, the tension cable runs through the flexible shaft and is pretensioned or pretensionable in the proximal direction at its proximal end via a pretensioning spring. Preferably, the pretensioning spring is connected to a tension roller around which the tension cable is deflected and connected at its distal end to a housing of the single-use operating portion. The tension roller and the pretensioning spring are in particular parts of the single-use operating part and are accommodated therein. In particular, the tension roller is attached to the housing of the single-use operating portion via a pretensioning spring in such a way that the tension roller is pretensioned or pretensionable in the proximal direction. The tension roller may thus pretension the tension cable in the proximal direction.

Preferably, the rotation mechanism is provided such that pulling on one of the control cables is converted into a rotational movement of the distal endoscope portion by a distal transducer or a distal transmission element (e.g. a gear train, a wrapped spool or the like). Further preferably, by releasing or loosening the corresponding control cable, the tension cable is pulled in the proximal direction by the pretensioning spring in such a way that the distal endoscope portion performs a reverse rotational movement.

Pretensioning of the tensioning spring is in particular greater than a resistance which acts against an axial movement of the tension cable and the control cable for controlling the rotation mechanism in the shaft.

Preferably, the single-use operating portion provides an air-water button configured to control an air and/or water supply to the distal endoscope portion.

In particular, the air-water button is connected to an air line forming an air supply line connecting the air-water button to a proximal air connection (e.g. in the processor port) and an air discharge connecting the air-water button to the distal endoscope head (e.g. via the roll hose and the fluid line in one of the control cables). The air supply line and the air discharge may be connected to each other directly or via the air-water button.

Furthermore, the air-water button may comprise a blow-off hole connected to the air line (in particular to the air supply line or at a transition area between the air supply and air discharge), through which the air line is opened to the environment. The blow-off hole is configured in particular such that it has a lower flow resistance than the air discharge.

The blow-off hole is configured in particular in such a way that it can be held closed by a user, e.g. by placing a finger on it. This makes it possible to ensure that compressed air, which is supplied to the operating portion via the air supply line, is either blown out through the blow-off hole or is directed to the endoscope head through the air discharge instead.

Preferably, the air-water button furthermore comprises a water switch, in particular a (micro) switch, which upon actuation activates a water supply to the endoscope head (e.g. via the roll hose and the fluid line in one of the control cables), e.g. by the water switch actuating a valve in a water supply line directly or via a control unit. Preferably, the microswitch is also configured to interrupt an air supply to the endoscope head when it is actuated, for example by actuating a valve in the air supply line directly or via a control unit or by the control unit interrupting a compressed air supply. The air-water button may therefore provide both a water and an air supply to the endoscope head in a simple, ergonomic way.

The air-water button also preferably has an actuation button or a grip plate in which the blow-off hole is configured. In particular, the actuation button or the grip plate is spring-mounted and superimposed on the water switch in such a way that it contacts the water switch via a (press) actuation of the user, thereby actuating it. If the actuation button or the grip plate is not actuated, the air-water button and possibly the control unit are configured not to activate or stop an air and water supply to the endoscope head. If the actuation button or the grip plate is actuated by holding the blow-off hole closed substantially without force or pressure, the air supply to the endoscope head is activated. If the actuation button or the grip plate is pressed against the water switch, a water supply to the endoscope head is actuated and preferably the air supply is interrupted. This allows the user to switch easily and intuitively between the air and water supply and switch them on or off as required.

Furthermore, the object underlying the present disclosure is solved by a reusable part (which has already been partially described above) of an/of the endoscope system having a reusable operating portion which is configured to be optionally coupleable with a single-use operating portion (in particular a single-use operating portion of the single-use part described above). The reusable part has a reusable housing and at least one electric drive (more precisely, electric motor) for controlling the endoscope system, which is accommodated in the reusable housing, and further has at least one reusable drivetrain portion (i.e. a power transmission cable) connected to the at least one electric drive to transmit a driving force to a longitudinally slidable reusable engagement element, wherein the at least one reusable drivetrain portion comprises the reusable engagement element configured for axially fixed coupling with a single-use drivetrain portion of the single-use operating portion. In particular, a distal end of the reusable drivetrain portion forms the reusable engagement element. More precisely, the reusable engagement element forms a free end of the reusable drivetrain portion in an uncoupled state.

In other words, the present disclosure relates to a reusable part of an endoscope system comprising at least one electric drive and a reusable drive train connected thereto, which comprises a distal output element. The distal output element is mounted so as to be slidable in a longitudinal direction, i.e. in the proximal and distal direction or toward and away from a single-use part attachable thereto, and in particular to be rotationally fixed. The reusable part furthermore has an electric drive which drives the distal output element (i.e. the reusable engagement element) in the longitudinal direction. The distal output element is configured to come into axially fixed engagement, in particular snap-in engagement, with a single-use drivetrain portion (a single-use engagement element) of a single-use part of the endoscope system. In particular, the reusable engagement element is configured such that, when a housing of the reusable part is coupled to a housing of the single-use part, in particular of the single-use operating part, it may be brought into and/or released from axially fixed engagement with the single-use drivetrain portion solely by actuation of the electric drive (i.e. without additional manual user actuation).

The reusable operating portion has, for example, components of the operating portion that are expensive to manufacture and/or are not or only laboriously recyclable, such as the electric drives. For example, the reusable operating portion forms a receptacle for the at least one electric drive, and optionally for at least one spindle drive described later, and may also have an operating block described later. The electric drive is in particular an electric motor, preferably a stepper motor.

The reusable drivetrain portion is in particular a mechanical and/or electrical (preferably purely mechanical) connection which is configured to transmit a driving force of the electric drive to a distal end of the reusable drivetrain portion or to the single-use drivetrain portion coupled thereto.

Preferably, the at least one reusable drivetrain portion has at least one thrust element which is axially slidably mounted in the reusable operating portion with respect to the shaft axis and has a distal end which forms the at least one reusable engagement element in particular in one piece of material or is connected thereto. Preferably, the at least one reusable drive train is arranged such that the at least one reusable engagement element protrudes distally from a reusable housing of the reusable operating portion in any position of the thrust element (i.e. in a most distal position up to a most proximal position which the thrust element can assume). This means that coupling of the reusable engagement element to the single-use drivetrain portion is performed outside of the reusable housing. This makes it possible to carry out the coupling within a single-use housing portion and to protect sensitive moving elements of the single-use drivetrain. Furthermore, the reusable housing may be sealed in any position of the thrust element and the reusable part is therefore particularly robust against internal contamination and resulting damage.

The thrust element is a longitudinally slidable element, in particular a longitudinally extending, i.e. tubular or rod-shaped element. The thrust element may preferably transmit thrust and tensile forces.

In particular, it is advantageous if the electric drive forms a brake or is configured to provide a braking effect, in particular in a switched-off or de-energized state. In this way, the electric drive may hold the thrust element in a retracted position, i.e. in the position furthest in the proximal direction. In this retracted position, the thrust element and possibly the reusable engagement element is preferably accommodated in the reusable housing for at least 70%, more preferably at least 80% or at least 90%, of its/their length. In this way, the reusable part can be provided, stored and transported individually, wherein the risk of damaging the reusable drivetrain portion is minimized.

Further preferably, the at least one reusable drivetrain portion comprises at least one spindle gear with at least one (threaded) spindle and at least one (spindle) nut arranged thereon rotationally fixed and axially slidable with respect to the housing, wherein the spindle nut is connected to the thrust element in order to drive it in the longitudinal direction. This is a particularly simple and reliable transmission gear for converting a rotary movement of the electric drive into a longitudinal movement of the thrust element. The thrust element is preferably mounted non-rotatably to the reusable housing, e.g. in that the thrust element and/or the spindle groove and/or a component connecting the spindle groove and the thrust element have a non-circular profile (e.g. polygonal/square/etc.).

A distal front side of one of the reusable housing has at least/in each case an opening for the at least one thrust element. A seal is provided between the opening and the respective thrust element in order to seal the reusable housing and to protect the components contained therein from dirt, moisture, etc. This may reduce or prevent contamination of an environment by abrasion from the reusable housing. Preferably, latching openings are also provided in the distal front side of the reusable housing, which are configured for latching noses of the single-use part, in particular the single-use operating part.

Preferably, a proximal end of the at least one spindle is connected to an output shaft of the at least one electric drive and comprises a spindle angle encoder, in particular in the form of an external toothing. Further preferably, a spindle angle receiver is provided adjacent to the spindle angle encoder (attached to the reusable housing), which detects a number of revolutions or an angle of rotation of the spindle. In particular, the angle encoder, e.g. in the form of a toothing, may be arranged on a proximal portion of the threaded spindle or a connecting sleeve which connects the threaded spindle and the output shaft of the drive. The at least one spindle angle receiver and the at least one spindle angle encoder may provide the number of revolutions or the angle of rotation of the spindle to a computer unit/a control unit of the endoscope system (or of an external unit) in order to calibrate the electric drive. This enables particularly precise control of the electric drive.

Preferably, the spindle is connected to an output shaft of the electric drive via an elastic friction element, e.g. an O-ring. The elastic friction element is preferably clamped between the spindle and the output shaft in order to transmit a torque of the electric drive between them. Furthermore, the elastic friction element is preferably configured in such a way that it slips at a predetermined torque, i.e. it acts as a safety clutch. This makes it possible to protect the electric drive, the single-use/reusable drivetrain portion and patient tissue from excessive strain if, for example, a movement of the distal endoscope portion is blocked or a part of the single-use/reusable drivetrain portion becomes jammed. This provides a particularly simple and cost-effective connection between the output shaft and the spindle. Furthermore, the elastic friction element may be configured in such a way that it compensates for an axial offset or misalignment between the output shaft and the spindle. Thus, tolerances in the production of the output shaft and the spindle and their assembly may be less precise, thereby reducing production and assembly costs.

Preferably, the spindle may be mounted on the reusable housing at its proximal end portion via two angular contact bearings (e.g. an X- or O-angled bearing) that are braced against each other. This provides particularly rigid mounting for the spindle and improves stability under high compressive and/or tensile loads or bending moments. Preferably, the spindle is furthermore mounted in the distal front side of the reusable housing, in particular by a slide bearing.

Preferably, the thrust element is a hollow tube which concentrically accommodates the spindle. In particular, a distal end of the thrust element may be integrally connected to or may form the reusable engagement element. As a result, a particularly uniform application of force to the thrust element can be achieved and the thrust element has a particularly high tensile and compressive strength. Another preferred feature is that the thrust element has the same outer contour as the reusable engagement element, at least at its distal end portion. This allows the thrust element to be retracted particularly far into the reusable housing, since a/the seal may seal the reusable housing both toward the thrust element and toward the distal coupling element. Alternatively, the thrust element may form a rod running parallel to the spindle, which may be non-circular, e.g. square.

Preferably, the thrust element is connected to a linear encoder. Further preferably, a linear receiver is arranged parallel to the tension and/or thrust element at least at a first end position and/or a second end position of a displacement movement of the thrust element, wherein the linear receiver is configured to detect when the linear encoder reaches the first end position or the second end position.

The linear encoder is preferably connected to the thrust element (a proximal end portion of the thrust element) or to a component that moves axially together with it (e.g. the spindle groove or a rotationally fixed entrainer/connector between the spindle groove and the thrust element). The linear encoder may be a marker, e.g. a magnet, a colored marker or the like. Preferably, the linear receiver is a component that detects the linear encoder, e.g. a Hall sensor, which detects when the linear encoder/magnet is positioned opposite to it. A signal from the linear receiver may be transmitted to a/the computer unit/control unit, which is configured to control the electric drive, in particular to stop the electric drive, when the thrust element reaches a position (in particular a distal and/or proximal end position) detected by the linear receiver. That is, the linear receiver may also be referred to as a switch position receiver/switch position sensor and the linear encoder may also be referred to as a switch position encoder.

This allows the thrust element to be driven between precisely defined end positions/end placement. This may advantageously prevent damage to the single-use and/or reusable drive train by overdriving the drive. For example, a first end position of a first or second thrust element may correspond to an extended position of a deflection or tilt head of the single-use part, in particular of the endoscope, and a second end position of the first or second thrust element may correspond to a most curved position of the deflection or tilt head of the single-use part, in particular of the endoscope. Furthermore, end positions of a third thrust element may correspond to a rotation mechanism twisted furthest in one or the other rotational direction.

The linear receiver (and possibly a circuit board carrying the linear receiver) are preferably arranged on a plane which is flat and parallel to the spindle. The reusable housing may, for example, have a reusable guide surface which is configured for contact with the single-use part, in particular the single-use operating part, in order to guide the latter, for example when the single-use part, in particular the single-use operating part, is plugged together or coupled with the reusable part. For example, the reusable guide surface may be a flat lateral side of the reusable housing. The reusable guide surface may be limited at its proximal end by a front surface perpendicular to it, on which an electrical reusable connection, and possibly guide pins or guide openings for guiding an axial relative movement (plug-in movement) between the single-use part, in particular the single-use operating part, and the reusable part, is/are provided. The linear receiver(s) may preferably be arranged on an internal surface of the reusable housing, which is opposite the reusable guide surface. In other words, a flat housing wall may form the guide surface on its outer side and support the linear receivers on its inner side. This allows the reusable housing to be configured in a particularly simple and ergonomic way.

The reusable part further preferably has an operating block, which has at least one manual operating element or operating wheel. The operating element or operating wheel is provided with an operation angle encoder. Further preferably, the reusable part has an operation angle receiver, which is arranged in the reusable housing adjacent to the operation angle encoder in order to detect a number of revolutions or an angle of rotation of the manual operating element or operating wheel and to provide this to a/the control unit/computer unit for controlling the electric drive element.

In particular, each electric drive is coupled to (exactly) one of the at least one manual operating element or operating wheel via a power and/or data connection. Preferably, each electric drive is controlled depending on, in particular proportional to, an input of a user at the manual operating element or operating wheel. Each manual operating element or operating wheel is preferably arranged on an outer side of the reusable housing of the reusable operating portion. Further preferably, each manual operating element or operating wheel is mounted on a shaft which is mounted on the reusable housing. If several manual operating elements or operating wheels are provided, the associated shafts are preferably mounted concentrically in each other.

Preferably, an operation angle encoder, in particular a magnetic disk (also referred to as an encoder wheel), is arranged on an inner end portion of each shaft, which rotates with the shaft. In addition, an operation angle receiver, in particular a Hall sensor, may be provided, which detects a number of revolutions or a rotation angle of the angle encoder or of the associated manual operating element or operating wheel. Each angle encoder is coupled in particular with (exactly) one of the electric drives/with the electric drive in order to control it. Preferably, a brake element, e.g. a brake plate or a separating disk or an O-ring or the like, is/are provided between the operating angle encoders, which mechanically separates the operating angle encoders from each other. In this way, each operating element/operating wheel may advantageously be braked so that it does not rotate unintentionally, e.g. due to a rotation of a neighboring operating element or operating wheel.

In particular, a plurality of electric drives and a plurality of reusable drivetrain portions are provided, which are each substantially independent of each other and which each comprise (exactly) one reusable engagement element, (exactly) one spindle gear with (exactly) one spindle and (exactly) one spindle nut, (exactly) one pull and/or thrust element. In this case, each of the reusable drivetrain portions is coupled or is coupleable to (exactly) one single-use strand portion and is coupled to (exactly) one of the electric drives. Similarly, a plurality of manual operating wheels or operating elements are provided, each of which is provided to operate/control (exactly) one of the electric drives.

Furthermore, the object underlying the present disclosure is solved by an endoscope system comprising a single-use part as described above and a reusable part as described above, which are selectively coupleable to each other, wherein the at least one reusable drivetrain portion and the at least one single-use drivetrain portion are selectively coupleable to respectively connect the at least one electric drive to the distal endoscope portion in order to control the distal endoscope portion.

In other words, an endoscope system is provided, comprising a (passive, driven) single-use portion and an (active, driving) reusable portion, in particular each as described above, wherein an output element of the reusable part is longitudinally movably supported for driving a single-use drivetrain portion. I.e. an output element of the drive train (power transmission cable) in the reusable part is an element that is slidable in a longitudinal direction. In particular, a transmission gear is provided for a transmission of a drive force, in particular in the form of a torque, into a longitudinal movement of a distal, controllable distal endoscope shaft portion in the reusable portion. A coupling mechanism for coupling the output element in the reusable part to an input element (single-use engagement element) of the drive train of the single-use part is configured to drive the output element of the reusable part longitudinally in order to bring the output element of the reusable part into axially fixed engagement with the input element of the drive train.

Preferably, the object is further solved by a method for coupling (coupling method) the single-use part described above and the reusable part described above. For this purpose, the single-use operating portion and the reusable operating portion are attached to each other, in particular guided or pushed along each other like drawers. Preferably, guide pins of the single-use part, in particular of the single-use operating part, or of the reusable part slide into guide holes of the respective other part (of the reusable or single-use part, in particular of the single-use operating part) during this attachment. Further preferably, latching noses of the single-use part, in particular of the single-use operating part, engage in latching openings of the reusable part when a coupled end position of housings of the single-use part, in particular of the single-use operating part, and of the reusable part is reached. Further preferably, during this attachment, an electrical connection of the single-use part, in particular of the single-use operating part, is connected to an electrical connection of the reusable part when the end position is reached. Subsequently, the at least one (preferably all) electrical drive is driven in a direction of thrust in order to push the thrust element out of the reusable housing, wherein it presses against the single-use engagement element in order to displace it in the distal direction until the single-use coupling portion abuts in the distal direction against its at least one stop. Subsequently, the at least one (preferably all) electric drive is driven further in the direction of thrust in order to advance the thrust element further in order to couple the reusable engagement element with the single-use engagement element, in particular in order to bring it into snap-in engagement.

Preferably, the at least one (preferably all) spindle gear is also calibrated via the spindle angle encoder and spindle angle receiver as well as the linear receiver and linear encoder, or a/the control unit/computer unit performs a corresponding calibration.

Furthermore, each drive is preferably driven depending on (in particular proportionally) a signal from (exactly) one operating angle receiver.

For decoupling, the at least one (preferably all) drive is preferably driven in a pulling direction in order to pull the single-use coupling portion in the proximal direction until it strikes against its at least one stop (a different stop than in the direction of thrust) in the proximal direction and is thereby held. Subsequently, preferably the at least one (preferably all) electric drive is driven further in the pulling direction in order to pull the thrust element further in the proximal direction in order to disengage the reusable engagement element from the single-use engagement element, i.e. to disengage, in particular to release a snap-in engagement thereof.

Furthermore, a system may be provided comprising the above-described reusable part (and optionally the above-described single-use part) and a computer unit configured to control the at least one electric drive in order to perform the above-described coupling method. In particular, the computer unit is connected to the at least one electric drive and/or the at least one operating angle receiver and/or the at least one spindle angle receiver and/or the at least one linear receiver. Preferably, the computer unit is integrated in the reusable part and may be supplied with power via the electrical reusable connection (i.e. via the processor port and the electrical single-use connection). Alternatively, the computer unit may be part of an external control unit and may be connected to the at least one electric drive and/or the at least one operating angle receiver and/or the at least one spindle angle receiver and/or the at least one linear receiver via the electric reusable connection (i.e. also via the processor port and the electric single-use connection).

### FIGURE DESCRIPTION

In the following, the present disclosure is described with reference to preferred embodiments. However, these are only illustrative in nature and are not intended to limit the scope of protection of the present disclosure. Furthermore, identical reference signs are used for the same components in the description of the various embodiments.

FIG. 1 shows a perspective view of an endoscope system according to the disclosure.

FIG. 2 shows a distal endoscope portion of the endoscope system according to FIG. 1 as well as a top view of an endoscope head of the endoscope system.

FIG. 3 shows a proximal operating portion of the endoscope system according to FIG. 1.

FIG. 4 shows a single-use operating portion of the operating portion according to FIG. 2.

FIGS. 5 and 6 each show different perspectives and a reusable operating portion of the operating portion according to FIG. 2, wherein a thrust element is modified differently in each case.

FIG. 7 shows an interior of the reusable operating portion according to FIG. 6.

FIG. 8 shows an operating block of the reusable operating portion according to FIGS. 5 and 6.

FIG. 9 shows a part of a drive train in the reusable operating portion according to the modification according to FIG. 5.

FIG. 10 shows the part of the drive train in the reusable operating portion according to the modifications according to FIG. 6.

FIG. 11 shows a further part of the drive train in the reusable operating portion according to the modifications according to FIG. 6.

FIG. 12 shows the further part of the drive train in the reusable operating portion according to the modification according to FIG. 5.

FIG. 13 shows a part of the single-use operating portion and a tension roller mounted therein.

FIG. 14 shows a cross-section through the single-use operating portion.

FIG. 15 illustrates a coupling or decoupling process between drive-train parts of the single-use operating portion and the reusable operating portion.

FIG. 16 shows a structure of an air-water button of the single-use operating portion.

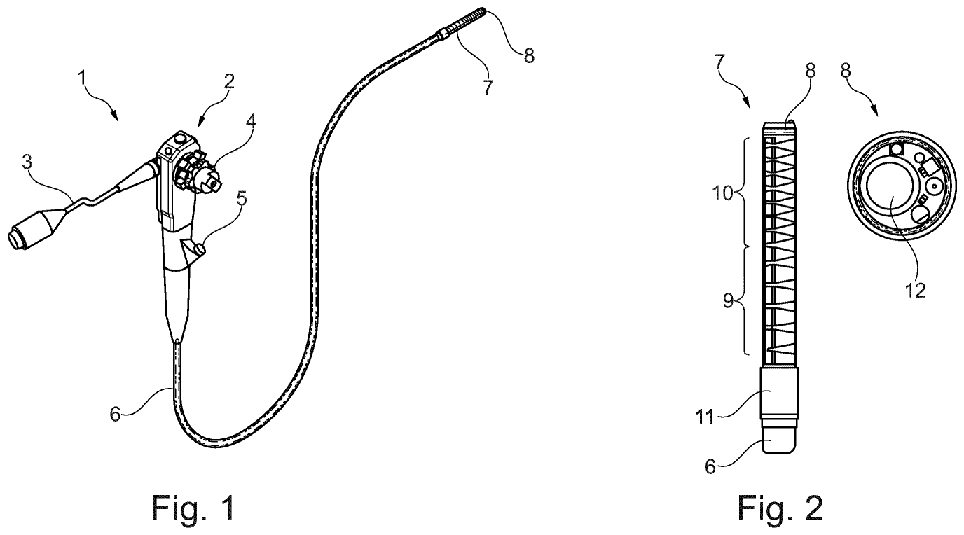

FIG. 1 shows a perspective view of an endoscope system 1 according to the disclosure. The endoscope system 1 shown has a proximal operating portion 2 (e.g. endoscope handle). The operating portion 2 has a processor connection 3, via which it is connected or connectable to a processor cable. Furthermore, the operating portion 2 has operating elements or wheels 4 and a connection 5, in particular a Luer connection. An endoscope, more precisely a flexible shaft 6 of the endoscope, extends in the distal direction from a distal end of the operating portion 2. A controllable distal shaft portion or endoscope portion 7, which is described in more detail with reference to FIG. 2, extends in the distal direction from a distal end portion of the flexible shaft 6. An endoscope head 8 is arranged at a distal end of the distal endoscope portion 7 or forms its distal end. A center axis of the flexible shaft 6 and of the distal endoscope portion 7 is referred to as the shaft axis.

As shown in FIG. 2, the distal endoscope portion 7 has a deflection 9, which is controllable or bendable to a first maximum curvature in order to pivot the endoscope head 8 in at least one or exactly one pivoting plane. Preferably, the distal endoscope portion 7 furthermore has a folding mechanism/tilt head 10, at the distal end of which the endoscope head 8 is formed. The folding mechanism/tilt head 10 is preferably arranged distally from the deflection 9 and can be controlled separately in order to swivel the endoscope head 8 in the swivel plane. Both the deflection 9 and the folding mechanism 10 preferably have several wedge-shaped segments, which are each hinged on their wide side to adjacent segments in such a way that they may be folded toward each other on their narrow side.

Preferably, a rotation mechanism 11 for rotating the distal endoscope portion 7 coaxially about the shaft axis is provided between the flexible shaft 6 and the distal endoscope portion 7. Furthermore, FIG. 2 shows a top view of the endoscope head 8. The endoscope head 8 forms an exit of a working channel 12 at its distal front side and carries further components such as a camera, a lighting equipment, a rinsing nozzle and the like.

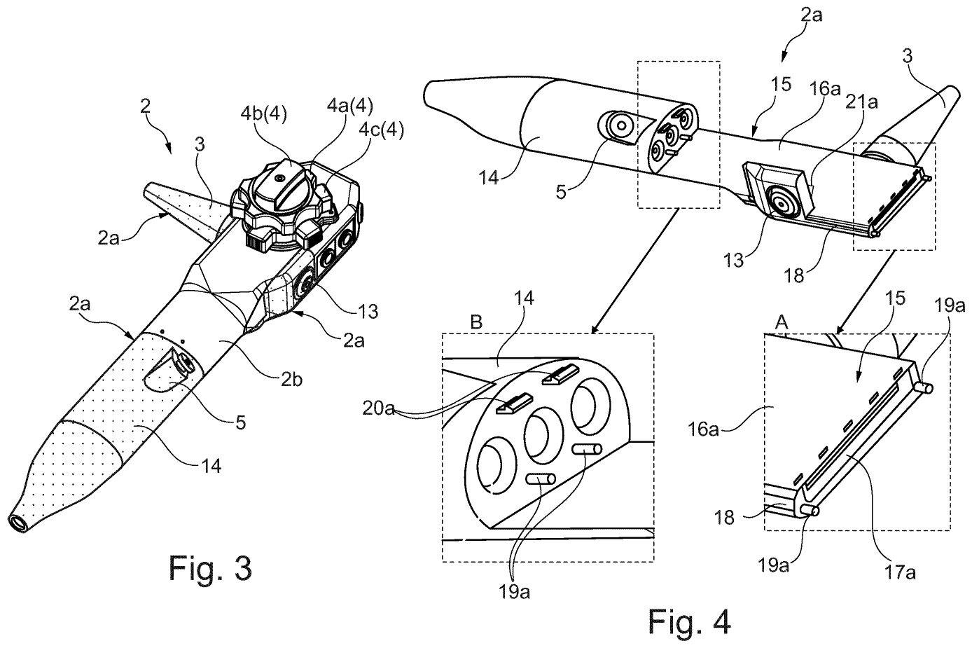

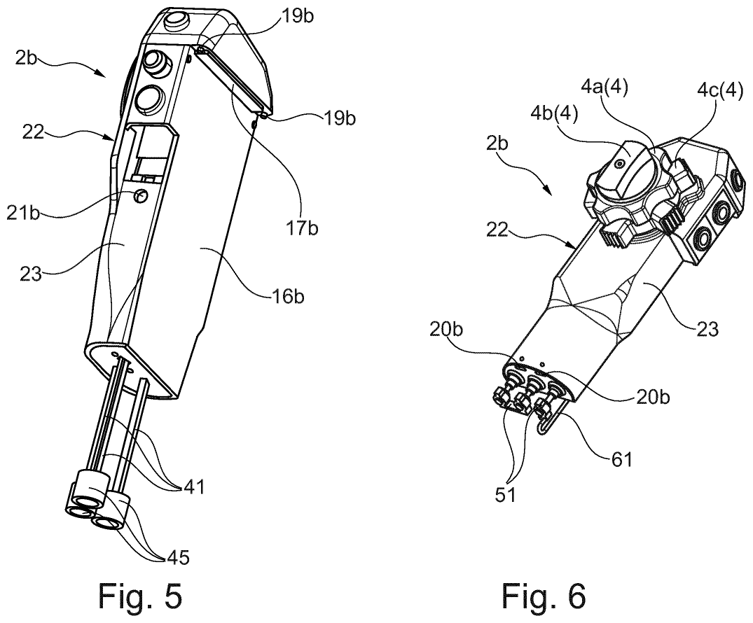

FIG. 3 shows the proximal operating portion 2 of the endoscope system 1 according to FIG. 1 in an assembled state. The operating portion 2 has a single-use operating portion 2a, which is shown in more detail in FIG. 4 (shown in dotted lines in FIG. 3), and a reusable operating portion 2b, which is shown in more detail in FIG. 5. The single-use operating portion 2a and the reusable operating portion 2b are coupled or coupleable to each other, in particular via a coupling mechanism according to the disclosure, which preferably has a plug-in mechanism.

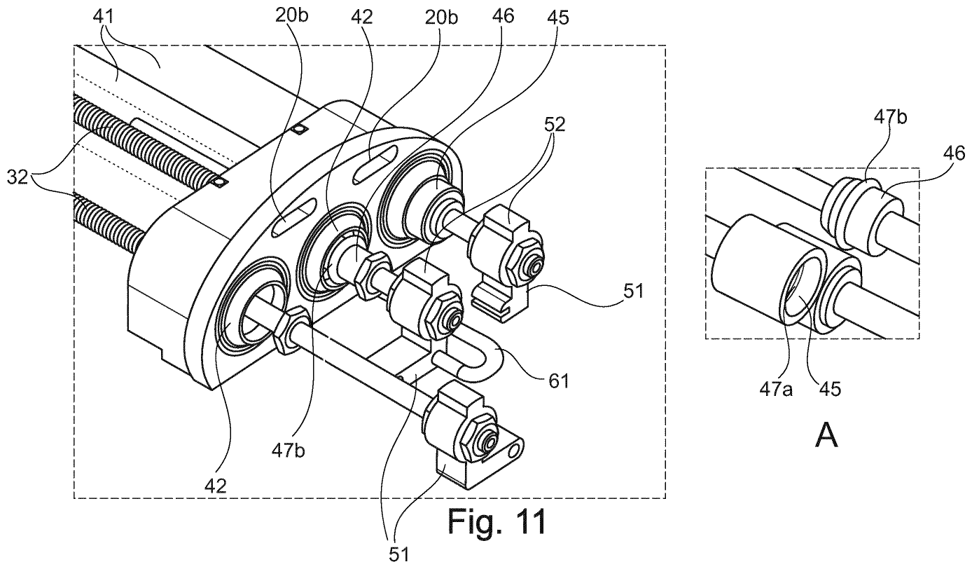

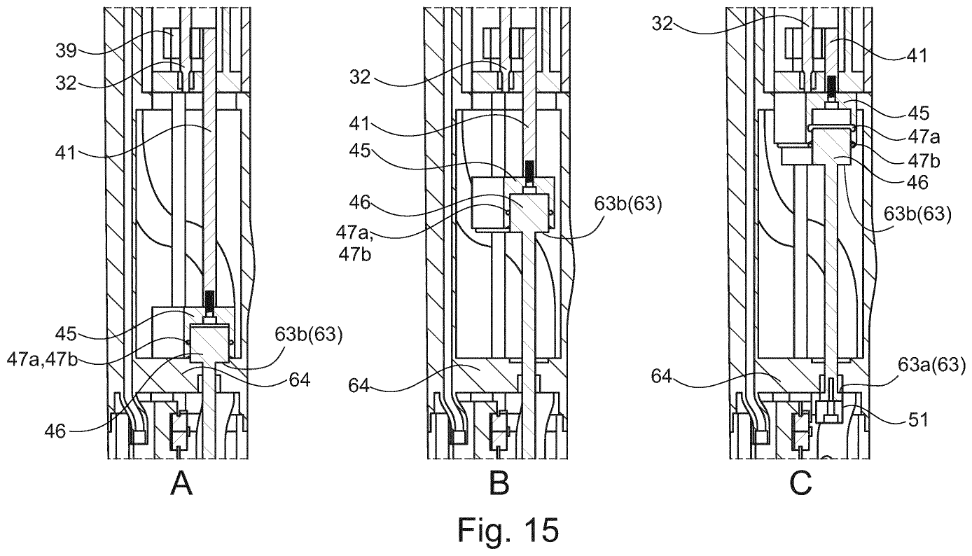

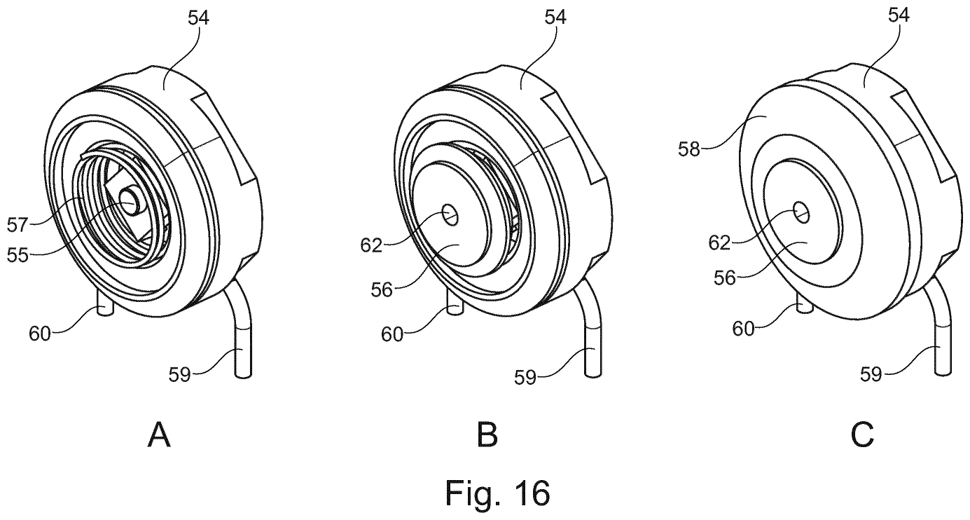

The single-use operating portion 2a is connected at its distal end to a proximal end portion of the flexible shaft 6. The distal end of the single-use operating portion 2a forms a substantially tubular or sleeve-shaped single-use housing portion 14, in the interior of which a connecting mechanism (see description below with reference to FIG. 11 to FIG. 15) is/are accommodated for connecting at least one, in particular three, control cable(s) 48 for controlling the distal endoscope portion 7 to the reusable operating portion 2b. At least one opening is formed on a proximal front side of the substantially tubular single-use housing portion 14, through which a drive-train portion of the single-use operating portion 2a is coupleable or decoupleable with a drive-train portion of the reusable operating portion 2b, which is described in more detail below with reference to FIG. 15 in particular.

As shown in FIG. 4, a plug portion 15 (a positioning plate) extends from the proximal front side of the single-use housing portion 14 in the proximal direction, preferably parallel to the shaft axis. Preferably, outer surfaces of the plug portion 15 and of the substantially tubular single-use housing portion 14 are continuously configured (i.e. aligned and connected without edges, in particular in one piece of material) on a radially outer side/lateral side with respect to the shaft axis. One surface of the plug portion 15, which is opposite the radial outer surfaces, forms a flat single-use guide surface 16a. This single-use guide surface 16a is configured for guidance or contact with a corresponding reusable guide surface 16b of the reusable operating portion 2b. Furthermore, labeling may be provided on the guide surface.

An air-water button 13 is preferably arranged on one side of the plug portion 15 with respect to the shaft axis, in particular on a protrusion which is located on a central area of the positioning plate with respect to the shaft axis. Lines connecting the air-water button 13 to the water connection (e.g. integrated in the processor port 3) and to the flexible shaft 6 are arranged within the single-use operating portion 2a, in particular within the protrusion and the plug portion 15. On a circumferential/lateral side of the single-use housing portion 14 with respect to the shaft axis, the (Luer) connection 5 is preferably provided and forms an access from an outer side of the single-use housing portion 14 to an interior of the single-use housing portion 14, in particular to a proximal end portion of the working channel 12.

A (in particular plug connector-like) electrical single-use connection 17a is configured on a proximal front edge of the plug portion 15 (see enlarged view A). This is configured for electrical coupling of the single-use operating portion 2a with a corresponding (in particular plug connector-like) electrical reusable connection 17b of the reusable operating portion 2b and further preferably for power transmission and possibly for signal transmission from the single-use operating portion 2a to the reusable operating portion 2b. The processor port 3 is preferably provided laterally on the plug portion 15 at a proximal end region thereof, i.e. close to the electrical single-use connection 17a. Cables connecting the processor port 3 and the electrical single-use connection 17a are accommodated in the plug portion 15.

Furthermore, the single-use operating portion 2a forms several mechanical plug elements, which are configured for the mechanical coupling of the single-use operating portion 2a with the reusable operating portion 2b (more precisely, housing parts thereof). At one or both side edges of the plug portion 15, i.e. an edge connecting the guide surface 16 and radial outer surface of the plug portion 15, an engagement rail 18 may be provided, which is configured to engage with a corresponding rail on the reusable operating portion 2b and to guide a displacement of the reusable operating portion 2b and of the single-use operating portion 2a relative to each other and parallel to the shaft axis.

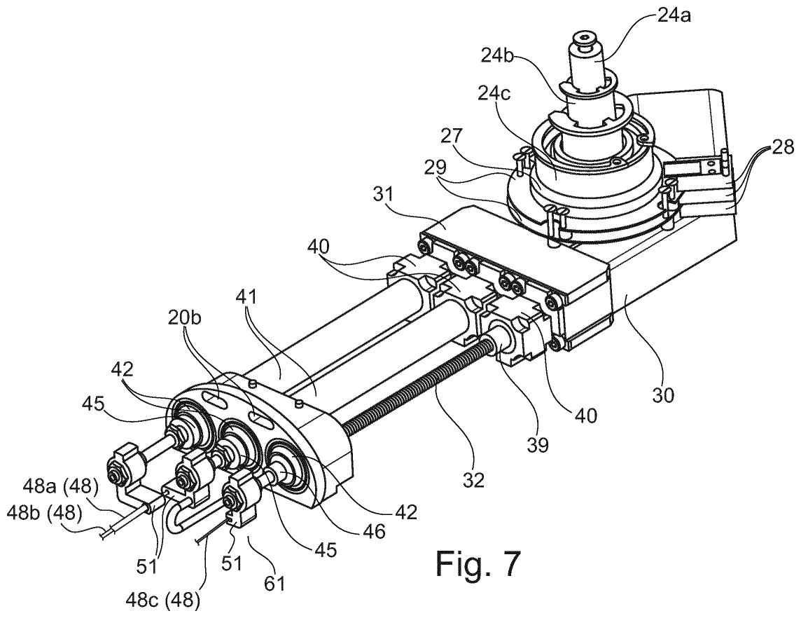

At least one, preferably a total of four, positioning openings 19b and/or positioning pins 19a, which are configured to engage with corresponding positioning pins 19a or positioning openings 19b (see FIG. 4, enlarged view A) on the reusable operating portion 2b, may be provided on the proximal front side of the plug portion 15, preferably on both sides of the electrical single-use connection 17a, and/or on the proximal front side of the substantially tubular single-use housing portion 14. Furthermore, latching noses 20a are preferably configured on the proximal front side of the single-use housing portion 14 (see FIG. 4, enlarged view B), which are configured for latching in latching openings 20b (see also FIG. 6 and FIG. 7) on the reusable operating portion 2b. Additionally or alternatively, on a radial inner side of the protrusion carrying the air-water button 13, a further latching spring 21a may be provided for latching to the reusable operating portion 2b, which is configured to latch into a further latching structure 21b of the reusable operating portion 2b.

Thus, for coupling the single-use operating portion 2a and the reusable operating portion 2b, the two portions (i.e. the single-use operating portion 2a and the reusable operating portion 2b) are placed on top of each other with the single-use guide surface 16a and the reusable guide surface 16b and, if applicable, the engagement rail 18 is brought into engagement with the rail of the reusable part 2b. The two portions 2a, 2b are then pushed toward each other/into each other parallel to the shaft axis in a plug-like/drawer-like manner until the positioning pins 19a, the latching noses 20a and the electrical single-use connection 17a come into engagement with the positioning openings 19b, latching openings 20b, and the electrical reusable connection 17b of the reusable part 2b. Once an end position is reached, the latching noses 20a and, if applicable, the latching spring 21a latch in the latching openings 20b and, if applicable, in the further latching structure 21b.

FIG. 5 and FIG. 6 each show the reusable operating portion 2b, wherein FIG. 5 shows a perspective view from the side of the reusable guide surface 16b and FIG. 6 shows a perspective view of an outer side of the reusable operating portion 2b.

The reusable operating portion 2b has a reusable housing 22, which has a substantially cuboid first notch on one side, which opens with respect to the shaft axis toward at least one, preferably both, lateral sides of the reusable housing 22 as well as toward a distal front side of the reusable housing 22. That is, in a side view, the notch defines a substantially L-shaped contour of the reusable housing 22. A bottom of the notch forms the reusable guide surface 16b and a proximal front side of the notch has the electrical reusable connection 17b and preferably the at least one, more preferably two, positioning opening(s) 19b. Alternatively or additionally, the at least one, preferably two, further positioning openings 19b are provided on the proximal front side of the reusable operating portion 2b or of the reusable housing 22. Preferably, the latching openings 20b are also provided on the distal front side.

In addition, the reusable housing 22 has a lateral contact surface 23, which is adjacent to the reusable guide surface 16b and extends substantially perpendicular thereto and parallel to the shaft axis. In particular, the reusable housing 22 forms a second substantially cuboid notch which opens at least in the distal direction and in the direction of the reusable guide surface 16b, wherein a bottom surface of the second notch forms the lateral contact surface 23. The lateral contact surface 23 is configured to come into contact with the protrusion carrying the air-water button 13. The further latching structure 21b may furthermore be configured in the lateral contact surface 23.

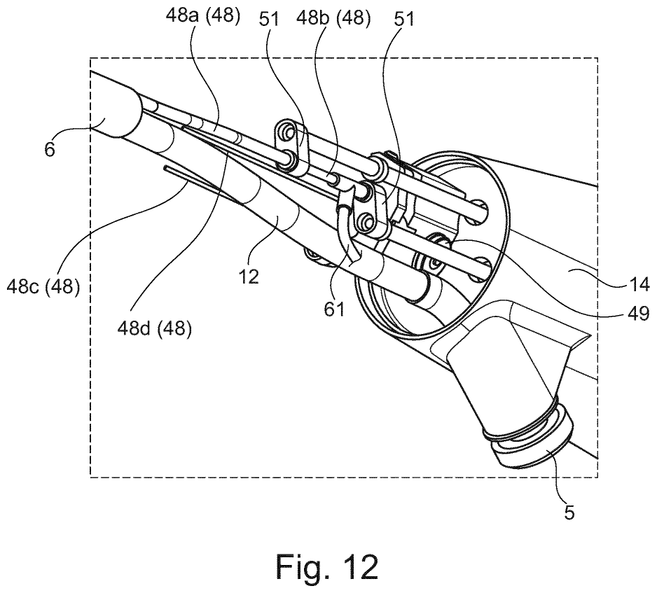

The distal front side of the reusable housing 22 furthermore has openings from which parts of a drive train for controlling the endoscope system 1 protrude. These are described in more detail below with reference to FIG. 11 and FIG. 12.

A plurality of operating elements or wheels 4 are provided on a side of the reusable housing 22 opposite the reusable guide surface 16b. In particular, the operating elements 4 are a plurality of concentric and rotatable handwheels. Preferably, a first operating element 4a is provided for actuating the folding mechanism/tilt head 10, a second operating element 4b for actuating the deflection 9 and a third operating element 4c for actuating the rotation mechanism 11.

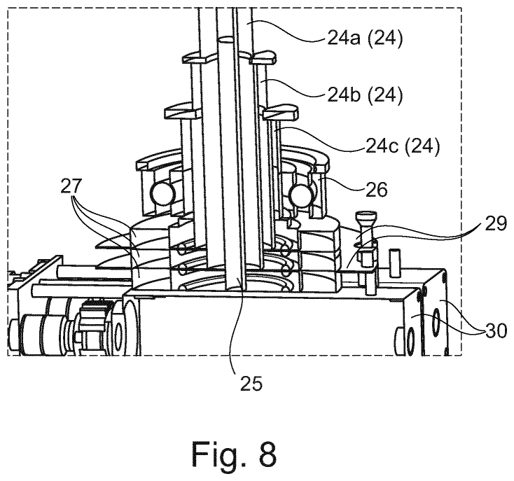

FIG. 7 shows an interior of the reusable operating portion 2b, in particular reusable drivetrain portions of the reusable operating portion 2b, which in FIG. 7 is already connected to components of a single-use drive train for illustration purposes. The drive-train portions of the reusable operating portion 2b have an operating block, which may be operated or handled by a user and is shown in more detail in FIG. 8.

The operating element block is described in more detail below with reference to a longitudinal sectional view of it shown in FIG. 8. In the present example, the operating elements 4 are each rotatably mounted via shafts 24 mounted concentrically in each other. That is, the outermost first operating element 4a (i.e. furthest away from the reusable housing 22) is fixed on a first shaft 24a, which is rotatably mounted on a central mandrel 25, which is fixed to the reusable housing 22. The middle, second operating element 4b is fixed on a second shaft 24b, which receives the first shaft 24a and is rotatably mounted thereto. The innermost third operating element 4b (i.e. closest to the reusable housing 22) is fixed on a third shaft 24c, which receives the second shaft 24b and is rotatably mounted relative thereto. A main bearing 26, in particular a roller bearing, is mounted on the outside of the third shaft 24c, which rotatably supports the three shafts 24a, 24b, 24c on the reusable housing 22.

The three shafts 24a, 24b, 24c are each firmly connected at their inner end portions to corresponding magnetic encoder wheels or operating angle encoders 27. Adjacent to the encoder wheels or operating angle encoders 27, a package of operating angle receivers 28 (see FIG. 7) or Hall sensors is arranged on the reusable housing 22, each of which detects a rotation (rotation angle/number of revolutions) of the magnetic encoder wheels or operating angle encoders 27 and converts it into an electrical signal. A respective brake element 29, such as a brake plate or a separating disk, is arranged between the adjacent magnetic encoder wheels or operating angle encoders 27, spacing the encoder wheels or operating angle encoders 27 from each other. The brake elements 29 have openings in which pins engage, which are connected to the reusable housing 22 in order to prevent the brake elements 29 from rotating relative to the reusable housing 22. The encoder wheels or operating angle encoders 27 may preferably be frictionally braked (e.g. via O-rings) on the brake element 29.

The angle receivers 28 are each connected to an electric drive 30 or electric motor in a data-transmitting manner, in particular via electric cables. The electric drives 30 and possibly the angle receivers 28 are preferably connected to the processor port 3 via the electrical single-use connections and reusable connections 17a, 17b and are supplied with power via these.

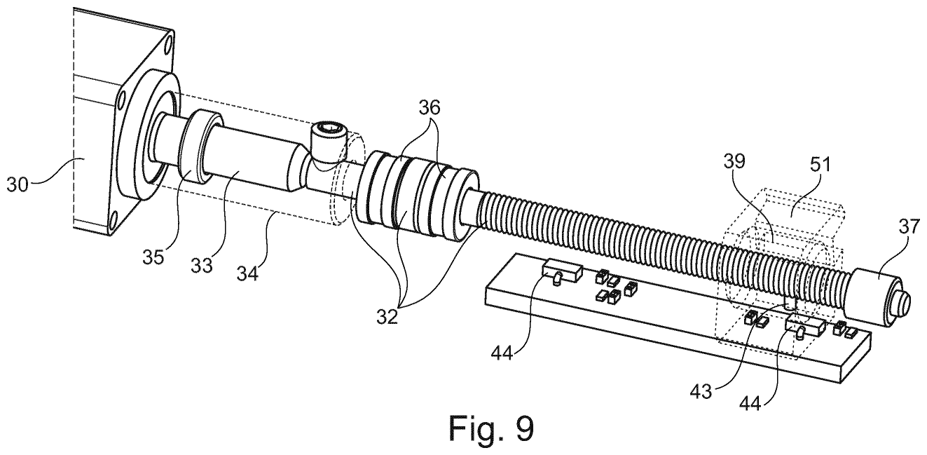

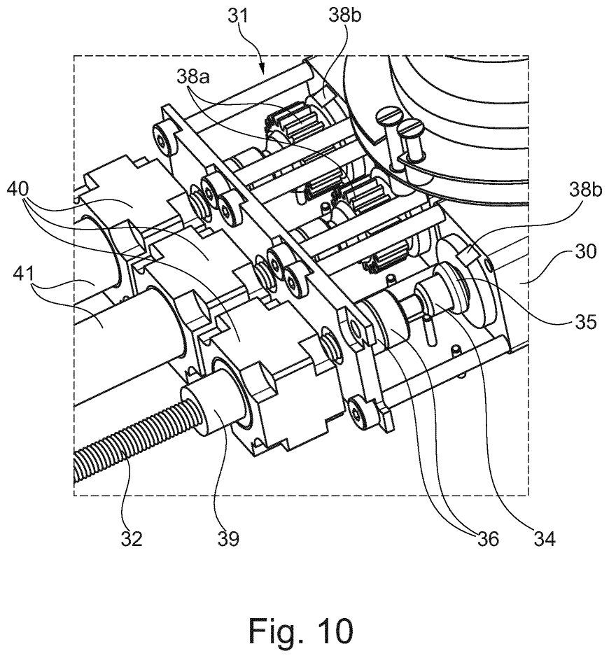

As shown in FIG. 7, each of the electric drives 30 is connected to a threaded spindle 32 via a spindle clutch block 31. This connection is described in more detail below with reference to FIG. 9 and FIG. 10. As shown in FIG. 9, the electric drive 30 (according to all modifications) has an output shaft 33. The output shaft 33 is immersed in a connecting sleeve 34. An elastic friction element 35, e.g. an O-ring, is clamped between the output shaft 33 and the connecting sleeve 34 in order to transmit a torque from the output shaft 33 to the connecting sleeve 34, to compensate for misalignment between the threaded spindle 32 and the output shaft 33, and to act as a torque limiter.

At its distal end portion, the connecting sleeve 34 is firmly connected to a proximal end portion of the threaded spindle 32, for example via a radial grub screw. Distal to the proximal end portion, the threaded spindle 32 is rotatable and axially fixed on the spindle clutch block 31 via thrust bearings or braced angular ball bearings 36, which is shown in a partially removed view in FIG. 10. The spindle clutch block 31 is firmly connected to the reusable housing 22. Furthermore, the threaded spindle 32 is preferably rotatably mounted at its distal end portion, in particular via a spindle bearing or plain bearing 37 (see FIG. 9).

Preferably, according to a modification (see FIG. 10), a spindle angle encoder 38a is provided proximally or distally of each of the thrust bearings or braced angular ball bearings 36, in particular in the form of a profile or a toothing, in particular an external toothing configured on the connecting sleeve 34. This spindle angle encoder 38a serves as a rotation-angle encoder, which interacts with a rotation-angle receiver arranged in or on the spindle clutch block 31 (shown here only individually by way of example) in order to detect a number of revolutions or a rotation angle of the spindle 32.

A nut 39 (shown here only as a dashed line), in particular a ball screw drive nut, is proximal to the spindle clutch block 31 on each of the threaded spindles 32. The nut 39 is connected, preferably directly or via a connector or entrainer 40 (shown only in dashed lines in FIG. 9), to a thrust element 41 in a rotationally fixed manner. The connector or entrainer 40 and/or the thrust element 41 is/are preferably non-circular, e.g. cuboid, in order to be rotationally fixed but axially slidingly mounted on the reusable housing 22. The connector or entrainer 40 and the thrust element 41 may each be directly connected to the nut 39. The thrust element 41 extends from the nut 39 or from the connector or entrainer 40 in a direction distally substantially parallel to the threaded spindle 32 and extends through one of the openings in the distal front side of the reusable housing 22. Preferably, the thrust element 41 is axially slidably guided or mounted in the distal front side of the reusable housing 22 or in a housing portion forming this front side, in particular via the plain bearing 37. Further preferably, a sealing 42 is preferably provided between the thrust element 41 and the distal front side of the reusable housing 22 (see FIG. 7, FIG. 11).

The thrust element 41 may be provided, for example, according to two different modifications of the embodiment, which are explained below.

In the embodiment shown in FIG. 7, the thrust element 41 is, for example, a thrust tube (see also FIG. 10 and FIG. 11) which concentrically accommodates the threaded spindle 32. FIG. 7 shows two of the drive-train portions with the thrust element 41 or thrust tube and one of the drive trains without the thrust element 41 or thrust tube to show the threaded spindle 32 accommodated therein and its attachment to the associated connector or entrainer 40. In this case, the threaded spindle 32 is preferably slidingly and rotationally mounted at its distal end portion in the thrust tube, in particular via the spindle bearing or plain bearing 37.

As a modification of the embodiment shown in FIG. 7, the thrust element 41 may be a push rod, as shown as an example in FIG. 5 and in FIG. 12 described below. In this case, the threaded spindle 32 is preferably mounted rotatably at its distal end portion in the distal front side of the reusable housing 22, e.g. via a slide bearing. Furthermore, the push rod is arranged axially offset and substantially parallel to the threaded spindle 32. The connector or entrainer 40 may have a lateral recess for inserting and fastening the push rod, e.g. according to the modification shown in FIG. 9. Optionally, a protective tube may be provided between the spindle clutch block 31 and the distal front side of the reusable housing 22, which protectively encloses the threaded spindle 32, as well as all or part of the connector or entrainer 40 and the push rod.

Preferably, a switch position encoder or (spindle) linear encoder 43, such as a permanent magnet, is mounted on a proximal end region of the thrust element 41 and/or on the nut 39 and/or (as shown, for example, in FIG. 9 as an example for all modifications) on the connector or entrainer 40. Furthermore, a sensor device may be arranged in the reusable housing 22 adjacent to the thrust element 41 (as shown, for example, in FIG. 9 as an example for all modifications), which has a corresponding switch position sensor or (spindle) linear receiver 44 at at least one position, which recognizes the switch position encoder or (spindle) linear encoder 43 when it has a predetermined or shorter distance from the switch position sensor or (spindle) linear receiver 44. Preferably, at least two switch position sensors or (spindle) linear receivers 44 are provided, which are located at positions which characterize a distal and a proximal end of a predetermined feed range/of a predetermined displacement movement of the thrust element 41. Preferably, the sensor device is provided on an inner side of the reusable housing 22 opposite the reusable guide surface 16b, since this is flat and the arrangement is therefore particularly simple.

Each thrust element 41 has a reusable engagement element 45, in particular a snap-fit sleeve, at its distal end portion. If the thrust element 41 is configured as the thrust tube, a distal end of the thrust tube may be configured as the reusable (snap-fit) coupling element 45. The reusable engagement element 45 forms an undercut configured to engage with a single-use engagement element 46 of a single-use coupling portion. As shown in particular in FIG. 7 and FIG. 11, the thrust elements 41 are preferably configured in such a way that at least the reusable engagement element 45 projects beyond the distal front surface of the reusable housing 22 in any position of the thrust element 41.

FIG. 7 and FIG. 11 furthermore show a part of the drive-train portion, which is assigned to the single-use operating portion 2a in each case and is already connected to the reusable engagement elements 45. A section of FIG. 7, in which individual parts (in particular a snap-fit sleeve and a snap-fit cylinder, as described below) are also hidden for better visualization, is shown in FIG. 11, to which reference is made below.

Preferably, one of the two engagement elements 45, 46 (i.e. the single-use engagement element 46 or preferably the reusable engagement element 45) forms a snap-fit sleeve with an inner circumferential wall in which a ring groove 47a or snap-fit groove is configured (see also FIG. 11, view A). Further preferably, the other one of the two engagement elements 45, 46 (e.g. the reusable engagement element 45 or preferably the single-use engagement element 46) forms a snap-fit cylinder with an outer circumferential wall in which an outer ring groove is configured. The snap-fit cylinder is dimensioned in such a way that it may be pushed into the snap-fit sleeve. An at least radially elastically deformable spring element 47b is held in the outer ring groove (see also FIG. 11, view A), in particular an annular spring element, such as a BalSeal-clutch spring. An outer diameter of the spring element 47b, which is held in the outer ring groove, in an uncoupled state is larger than an inner diameter of the inner circumferential wall of the snap-fit sleeve or of the snap-fit cylinder. For connecting the two engagement elements 45, 46, for example, the snap-fit cylinder is pushed into the snap-fit sleeve, wherein the spring element 47b is elastically deformed radially inward until it reaches the ring groove 47a and relaxes therein again in order to engage in the ring groove 47a and thus connect the two engagement elements 45, 46 to each other. An automatic coupling or decoupling method for connecting the two engagement elements 45, 46 is described in more detail below with reference to FIG. 15.

On its distal side, each of the single-use engagement elements 46 is connected to one of the control cables 48, as described with reference to FIG. 11 to FIG. 15. A part of the single-use drivetrain portions, which extends from the single-use engagement element to a proximal end portion of the control cable 48, is referred to as a single-use coupling element. More precisely, in the present embodiment, a first single-use coupling element is connected to a deflection control cable 48a, a second single-use coupling element is connected to a tilt head control cable 48b and a third single-use coupling element is connected to a rotation mechanism control cable 48c, which accordingly transmit an actuating force to the folding mechanism/tilt head 10, the deflection 9, and the rotation mechanism 11.

Preferably, the tilt head-control cable 48b is configured as a low-stretch tube, which is configured to transmit at least tensile forces, preferably also compressive forces, to the folding mechanism/tilt head 10 for its control. Furthermore, the tilt head control cable 48b forms a fluid channel in its interior, in particular a lens cleaning channel for cleaning the camera, which opens at a distal front side of the endoscope head 8. Further preferably, the deflection control cable 48a is configured as a low-stretch tube, which is configured to transmit at least tensile forces, preferably also compressive forces, to the deflection 9 for its control. The deflection control cable 48a extends in particular coaxially to the tilt head control cable 48b and accommodates the latter.

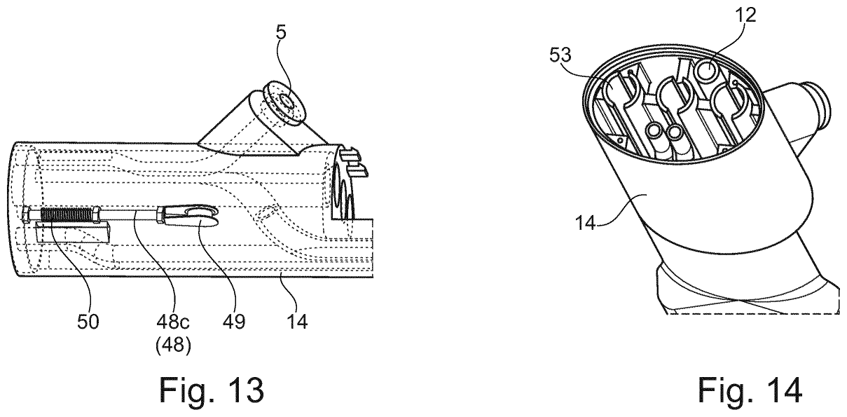

Further preferably, the rotation mechanism-control cable 48c is a cable which is configured to transmit tensile forces to the rotation mechanism 11 for its control. In particular, the rotation mechanism-control cable 48c is guided distally from the operating portion 2 through the flexible shaft and is connected at its distal end to a distal transducer, which is configured to convert the control cable movement into a rotation of the distal endoscope portion 7. The transducer is connected to a tension cable 48d, which extends proximally through the flexible shaft 6 and is attached pretensioned to the single-use housing portion 14 in a proximally direction. The tensioning of the tension cable 48d is achieved via a tension roller 49, which (as shown in FIG. 13) is accommodated in the single-use housing portion 14. The tension roller 49 is both rotatable and axially (along the shaft axis) slidable, e.g. via a slide, and is pretensioned in the proximal direction via a cable tensioning device 50 or tension spring. The cable tensioning device 50 according to FIG. 13 preferably has a compression spring.

Preferably, cross connectors 51 are provided to connect the single-use engagement elements 46 to the respective associated control cables 48a, 48b, 48c (see FIGS. 7, 11 and 12) and to compensate for a transverse offset therebetween. For example, one of the cross connectors 51 is connected to one of the single-use engagement elements 46 and forms a clamping receptacle axially offset thereto, which is configured to hold the rotation mechanism control cable 48c in a clamping manner. A further one of the cross connectors 51 is connected to a further one of the single-use engagement elements 46 and is connected axially offset thereto to the deflection control cable 48a, which preferably holds the tilt head control cable 48b. A further one of the cross connectors 51 is connected to a further one of the single-use engagement elements 46 and is connected axially offset thereto to the tilt head-control cable 48b, which preferably forms the fluid channel.

The cross connector 51, which connects the tilt head control wire 48b to the corresponding single-use engagement element 46, may also serve as a fluid line adapter (see FIG. 7 and FIG. 11) in order to connect a fluid line, which is fed from the processor port 3 through the single-use operating portion 2a, to the tilt head control cable 48b serving as a fluid line. For this purpose, the corresponding cross connector 51 forms a fluid connection, to which the fluid line coming from the processor port 3 is connected, as well as a feed-through, which connects the fluid connection to the tilt head-control cable 48b. A distal portion of the fluid line coming from the processor port 3 is configured in particular as a flexible roll hose 61, so that longitudinal displacement of the tilt head-control cable 48b is possible.

As an alternative to the modification shown in FIG. 11, both the tilt head control cable 48a and the deflection control cable 48b may, as shown in FIG. 12, be connected to the associated single-use engagement elements 46 in an axially offset manner via a cross connector 51 in each case. Furthermore, as shown in FIG. 12, the fluid connection for the connection of the fluid line coming from the processor port 3 as may be a component provided separately from the cross connectors 51 and connected to the tilt head-control cable 48a.

Furthermore, the cross connectors 51 are preferably non-circular or profiled. For example, the cross connectors 51 have cross-pieces 52 projecting radially with respect to the longitudinal direction, which are provided as an anti-twist device of the cross connectors 51 in the single-use housing portion 14 (see FIG. 7 and FIG. 11). The single-use housing portion 14 forms corresponding axial guides 53 (see FIG. 14) with grooves in its interior, in which the cross connectors 51 engage in a rotationally fixed manner and are guided in an axially displaceable manner. This can be clearly seen in FIG. 14, which shows a cross-section of the single-use housing part 14. The single-use housing part 14 may also form further lines, e.g. an air line and a water line, as well as a proximal portion of the working channel 12, in particular in one piece of material, which are shown in cross-section in FIG. 14.

FIG. 15 illustrates an automatic decoupling process of the engagement elements 45, 46. Furthermore, FIG. 15 serves to illustrate an automatic coupling process, which is preferably triggered automatically when the computer unit recognizes that the single-use operating portion 2a and the reusable operating portion 2b have been plugged together, e.g. by closing the electrical connections 17a, 17b or a switch.

FIG. 15 shows several sections of a longitudinal cross-section of the endoscope 1 with the single-use and reusable operating portions 2a, 2b plugged together in the area of the single-use housing portion 14, wherein only one of the drive-train portions is shown in more detail as an example. In this assembled state, the thrust element 41 and the reusable engagement element 45 are arranged at least partially within the single-use housing portion 14, more precisely in a proximal chamber in the single-use housing portion 14. The single-use housing portion 14 is divided into a proximal and a distal chamber by a partition wall or a frame 64. The single-use coupling portion of the single-use operating portion 2a extends axially slidably through the partition wall or frame 64 such that the single-use engagement elements 46 are accommodated in the proximal chamber and the cross connector 51 and the control cable 48 are arranged in the distal chamber. The partition wall or frame 64 is configured in such a way that stop portions 63 of the connecting mechanism may come into supporting contact with it.

In view A of FIG. 15, the single-use engagement elements 46 and the reusable engagement elements 45 are coupled. The thrust elements 41 are moved in the distal direction. In order to separate the engagement elements 45, 46, the drives (in particular all drives) are actuated in order to retract the thrust elements 41 (i.e. to move in a proximal direction), as shown in view B. At a predetermined travel distance, proximal stop portions 63a of the stop portion 63 abut on the partition wall or frame 64 of the single-use housing portion 14 (see view C). In particular, the proximal stop portions 63a are provided on the single-use coupling portions between the single-use engagement elements 46 and the cross connectors 51, e.g. configured as adjustment stop components, such as adjusting nuts. Alternatively, the proximal stop portion 63a may be provided, for example, by one of the cross connectors 51 or by a stop disk or step attached to the connecting rod. If the thrust elements 41 are then moved further in the proximal direction, as shown in view C, pressure is exerted on the radially elastically deformable spring elements 47b to such an extent that they deform and become disengaged from the reusable engagement elements 45. This releases the connection between the single-use and reusable engagement elements 45, 46.

In the opposite direction, i.e. starting from view C of FIG. 15, the thrust elements 41 are pushed forward in the distal direction. In doing so, they push the single-use engagement elements 46 in front of them until they abut against the proximal front side of the single-use housing portion 14. In this case, the single-use engagement elements 46 or their distal front surfaces each serve as the distal stop portion 63b of the stop portion 63. When the thrust elements 41 are then moved further in the distal direction, as shown in view A, pressure exerted on the radially elastically deformable spring elements 47b so high that they deform and each move into a gap between the single-use and the reusable engagement elements 45, 46 until they snap into the ring grooves 47a of the reusable engagement elements 45. This creates the connection between the single-use and reusable engagement elements 45, 46.

FIG. 16 illustrates the structure of the air-water button 13 shown in FIG. 4. The air-water button 13 has a base body 54. This may be a separate part or may be configured integrally with the protrusion on the plug portion 15 of the single-use operating portion 2a. As can be seen in FIG. 16, section A, a recess is configured in the base body 54, in which a microswitch 55 is arranged. The microswitch 55 is, for example, a pressure switch or touch sensor. A grip plate or an actuation button 56 (see FIG. 16, section B) is positioned in front of the microswitch 55 via a spring 57 and is preferably attached to the base body 54 via a flexible membrane 58 (e.g. a rubber cap) (see FIG. 16, section C). The flexible membrane 58 may serve as the spring or, as in the present example, an additional spring 57 may be provided.

The grip plate or the actuation button 56 is mounted on the base body 54 in such a way that it may be brought into contact with the microswitch 55 by actuation of a user against a pre-stressing of the spring 57 in order to actuate the latter. The microswitch 55 is connected to a computing unit which is configured to activate a flush function of the flushing device on the endoscope head 8 when the microswitch 55 is actuated, for example by configuring the computing unit to activate an external pump which is connected to the endoscope system 1 via a flush line. The flush line may, for example, be connected to flush lines in the operating portion 2 of the endoscope system 1 via the processor port 3.

Furthermore, the air-water button 13 is connected to an air supply line 59 and an air discharge 60, which are connected to each other. The air discharge 60 is connected to the endoscope head 8. The air discharge 60 may optionally be connected to a line which is configured substantially completely fixed in position in the single-use housing part 14 and in the plug portion 15, or it may be connected to the roll hose 61 described above. The air supply line 59 is connected in particular to the processor port 3 and may be supplied with compressed air via it. In addition, a blow-off hole 62 is provided in the grip plate or the actuation button 56, which connects the environment with the air discharge 60 and the air supply line 59. For example, the blow-off hole 62 is directly connected to the air discharge and air supply line 59, 60 or both the air discharge and air supply line 59, 60 and the blow-off hole 62 open into a space between the walls of the trough and the flexible membrane 58. The blow-off hole 62 is configured and dimensioned in particular in such a way that it may be easily held shut or closed by the user. Furthermore, the opening is configured in such a way that a flow resistance of the blow-off hole 62 is smaller than a flow resistance of the air discharge 60. This means that compressed air, which is applied to the air supply line 59, only flows through the air discharge 60 when the blow-off hole 62 is held closed. If the user presses harder on the grip plate or the actuation button 56, the microswitch 55 is actuated, thereby activating the flush function. Optionally, the air-water button 13 or the computing unit is also configured to interrupt the air supply to the air discharge 60 when the microswitch 55 is actuated, e.g. by interrupting a connection between the air supply line 59 and the air discharge 60 in the manner of a valve.

It should be noted that although the embodiment described above provides several drive lines for the actuation of several different functions (of the tilt head 10, deflection 9, and rotation mechanism 11), these may be independent of each other and thus only one or any two of these drive lines may be provided. It should also be noted that, although the above description often refers to only one of the drive trains, the other two drive trains may be substantially configured in the same way, apart from the differences described above. It should also be noted that the above description refers to modifications of the endoscope according to the disclosure in which only individual, explicitly described components differ from the embodiment.

List of reference signs

| | |
|---|---|
| 1 | endoscope system |
| 2 | operating portion or endoscope handle |
| 2a | single-use operating portion |
| 2b | reusable operating portion |
| 3 | processor port |
| 4 | operating element or wheels |
| 4a, b, c | first/second/third |
| | operating element |
| 5 | (Luer) connection |
| 6 | flexible shaft |
| 7 | distal endoscope portion |
| 8 | endoscope head |
| 9 | deflection |
| 10 | folding mechanism |
| 11 | rotation mechanism |
| 12 | working channel |
| 13 | air-water button |
| 14 | single-use housing portion |
| 15 | plug portion |
| 16a | single-use guide surface |
| 16b | reusable guide surface |
| 17a | electrical single-use connection |
| 17b | electrical reusable connection |
| 18 | engagement rail |
| 19a | positioning pins |
| 19b | positioning openings |
| 20a | latching noses |
| 20b | latching openings |
| 21a | latching spring |
| 21b | further latching structure |
| 22 | reusable housing |
| 23 | lateral contact surface |
| 24 | shafts |
| 24a, b, c | first/second/third shaft |
| 25 | central mandrel |
| 26 | main bearing |
| 27 | encoder wheels/operating angle |
| | encoders |
| 28 | operating angle receiver |
| 29 | brake elements/separating discs/ |
| | brake plates |
| 30 | electric drive/electric motor |
| 31 | spindle clutch block |
| 32 | (threaded) spindle |
| 33 | output shaft |
| 34 | connecting sleeve |
| 35 | elastic friction element/O-ring |
| 36 | thrust bearing/braced angular |
| | contact bearing |
| 37 | spindle bearing/slide bearing |
| 38a | spindle angle encoder/toothing |
| 38b | spindle angle receiver/toothing |
| | sensor |
| 39 | (spindle) nut |
| 40 | connector/entrainer |
| 41 | thrust element/thrust tube |
| 42 | sealing |
| 43 | switch position encoder/(spindle) |
| | linear encoder |
| 44 | switch position sensor/(spindle) |
| | linear receiver |
| 45 | reusable engagement element/ |
| | snap-fit sleeve |
| 46 | single-use engagement element/ |
| | snap-fit cylinder |
| 47a | ring groove/snap-fit groove |
| 47b | radially elastically deformable |
| | spring element |
| 48 | control cable |
| 48a | deflection control cable |
| 48b | folding-mechanism control cable/ |
| | tilt-head control cable |
| 48c | rotation mechanism-control cable |
| 48d | tension cable |
| 49 | tension roller |
| 50 | cable tensioning device/tension |
| | spring |
| 51 | cross connector |
| 52 | cross-pieces/anti-twist device |
| 53 | axial guides |
| 54 | base body |
| 55 | microswitch |
| 56 | actuation button/grip plate |
| 57 | spring |
| 58 | flexible diaphragm |
| 59 | air supply line |
| 60 | air discharge |
| 61 | roll hose |
| 62 | blow-off hole |
| 63 | stop portion |
| 63a | proximal stop element |
| 63b | distal stop element |
| 64 | partition wall or frame |

1-15. (canceled)

16. A single-use part of an endoscope system comprising
a single-use operating portion configured to be selectively coupled to a reusable part of the endoscope system,
an endoscope including:
(i) a flexible shaft, which is configured for insertion into a body opening of a patient, wherein the flexible shaft is connected to the single-use operating portion and extends therefrom along a shaft axis in a distal direction,
(ii) a distal endoscope portion connected to a distal end portion of the flexible shaft, and
(iii) a distal endoscope head forming a distal front side of the distal endoscope portion or being attached thereto, and
at least one single-use drivetrain portion connecting the single-use operating portion to the distal endoscope portion in order to control the distal endoscope portion,
wherein the at least one single-use drivetrain portion has at least one single-use coupling portion, which is slidably mounted in or on the single-use operating portion in a direction of the shaft axis and has a single-use engagement element configured at a proximal end thereof, which is configured for axially fixed coupling with a reusable drivetrain portion of a reusable operating portion.

17. The single-use part of the endoscope system according to claim 16, wherein:
the single-use operating portion forms a distal single-use housing portion, in which the at least one single-use coupling portion is mounted so as to be slidable in a longitudinal direction, and
a corresponding displacement movement in at least one direction along the shaft axis is limited by a stop, in such a way that the single-use engagement element is located in a most proximal position at least partially within the distal single-use housing portion.

18. The single-use part of the endoscope system according to claim 17, wherein the single-use housing portion forms a proximal front side, in which at least one opening for insertion of the reusable drive train portion is configured, and forms a plug portion, which extends in a proximal direction from the single-use housing portion and forms an electrical single-use connection on a proximal front surface thereof.

19. The single-use part of the endoscope system according to claim 16, wherein the single-use operating portion forms a latching nose configured for snap-in engagement and has a predetermined breaking point configured to break when the snap-in engagement is released.

20. The single-use part of the endoscope system according to claim 16, wherein the at least one single-use coupling portion is connected at a distal end thereof to a cross connector with a respective control cable, wherein the cross connector compensates for a radial offset.

21. The single-use part of the endoscope system according to claim 20, wherein:
the control cable is a low-stretch tube in which a fluid channel is configured, and
an associated cross connector forms a connection for a flexible roll hose and fluidically connects the flexible roll hose to the fluid channel in the control cable.

22. The single-use part of the endoscope system according to claim 20, wherein the control cable runs through the flexible shaft and is connected at a distal end thereof to a tension cable which runs through the flexible shaft and is deflected at a proximal end thereof via a resiliently pretensioned tension roller and is connected to a housing of the single-use operating portion, wherein the tension roller is attached to the housing of the single-use operating portion via a tension spring in such a way that the tension roller is pretensioned or pretensionable in a proximal direction.

23. The single-use part of the endoscope system according to claim 16, wherein the single-use operating portion includes an air-water button which is connected to an air line forming an air supply line connecting the air-water button to a proximal air connection and an air discharge connecting the air-water button to the distal endoscope head, wherein the air-water button further comprises a blow-off hole connected to the air line, through which the air line is opened to the environment and which is configured to have a lower flow resistance than the air discharge.

24. A reusable part of an endoscope system, said reusable part comprising:
a reusable operating portion (i) configured to be selectively coupleable with a single-use operating portion, and (ii) comprising a reusable housing and at least one electric drive for controlling an endoscope of the endoscope system, the at least one electric drive being located within the reusable housing, and
at least one reusable drivetrain portion connected to the at least one electric drive for transmitting a driving force to a reusable engagement element,
wherein the at least one reusable drivetrain portion comprises the reusable engagement element, which is mounted longitudinally slidably on the reusable housing and which is configured for axially fixed coupling with a single-use drivetrain portion of the single-use operating portion.

25. The reusable part of the endoscope system according to claim 24, wherein the at least one reusable drivetrain portion comprises at least one spindle gear with at least one spindle and at least one nut arranged thereon rotationally fixed and longitudinally slidable with respect to the reusable housing, which is connected to a thrust element, wherein the thrust element is longitudinally slidably mounted in the reusable operating portion and has a distal end which forms the at least one reusable engagement element.

26. The reusable part of the endoscope system according to claim 25, wherein the thrust element is a hollow tube which concentrically accommodates the spindle and which has a same outer contour as the reusable engagement element.

27. The reusable part of the endoscope system according to claim 25, wherein:
the thrust element is connected to a linear encoder, and
a linear receiver is arranged parallel to the thrust element at least at a first end position and a second end position of a displacement movement of the thrust element, wherein the linear receiver is configured to detect when the linear encoder reaches either the first end position or the second end position.

28. The reusable part of the endoscope system according to claim 25, wherein:
a proximal end of the at least one spindle is connected to an output shaft of the at least one electric drive and comprises a spindle angle encoder in the form of an external toothing, and
a spindle angle receiver is provided adjacent to the spindle angle encoder, which detects a number of revolutions or an angle of rotation of the spindle for controlling and/or calibrating the electric drive.

29. The reusable part of the endoscope system according to claim 24, further comprising:
an operating block, which has at least one manual operating element or operating wheel, which is provided at an inner end thereof with an operation angle encoder, and
an operation angle receiver, which is arranged in the reusable housing adjacent to the operation angle encoder in order to detect a number of revolutions or an angle of rotation of the manual operating element or operating wheel in order to make it available for controlling the electric drive.

30. An endoscope system comprising:
(A) a single-use operating portion comprising (a) an endoscope including:
(i) a flexible shaft, which is configured for insertion into a body opening of a patient, wherein the flexible shaft is connected to the single-use operating portion and extends therefrom along a shaft axis in a distal direction,
(ii) a distal endoscope portion connected to a distal end portion of the flexible shaft, and
(iii) a distal endoscope head forming a distal front side of the distal endoscope portion or being attached thereto, and
(b) at least one single-use drivetrain portion connecting the single-use operating portion to the distal endoscope portion in order to control the distal endoscope portion, wherein the at least one single-use drivetrain portion has at least one single-use coupling portion, which is slidably mounted in or on the single-use operating portion in a direction of the shaft axis and has a single-use engagement element configured at a proximal end thereof; and
(B) a reusable operating portion comprising (i) a reusable housing and at least one electric drive for controlling the endoscope of the endoscope system, the at least one electric drive being located within the reusable housing, and (ii) at least one reusable drivetrain portion connected to the at least one electric drive for transmitting a driving force to a reusable engagement element, wherein the at least one reusable drivetrain portion comprises the reusable engagement element, which is mounted longitudinally slidably on the reusable housing and which is configured for axially fixed coupling with the single-use drivetrain portion of the single-use operating portion,
wherein the reusable engagement element of the at least one reusable drivetrain portion and the single-use engagement element of the at least one single-use drivetrain portion are selectively coupleable to respectively connect the at least one electric drive to the distal endoscope portion.
